(19) United States
(12) Patent Application Publication
(10) Pub. No.: US 2022/0229059 A1
(43) Pub. Date: Jul. 21, 2022

(54) TUMOR CELL-DERIVED EXOSOMES AND THEIR APPLICATIONS

(71) Applicants: The General Hospital Corporation, Boston, MA (US); University of Pittsburgh - Of the Commonwealth System of Higher Education, Pittsburgh, PA (US)

(72) Inventors: Soldano FERRONE, Boston, MA (US); Theresa L. WHITESIDE, Pittsburgh, PA (US)

(21) Appl. No.: 17/615,410

(22) PCT Filed: Jun. 3, 2020
(86) PCT No.: PCT/US2020/035898

§ 371 Date: Nov. 30, 2021

Related U.S. Application Data
(60) Provisional application No. 62/856,195, filed on Jun. 3, 2019.

(51) Int. Cl. G01N 33/574; G01N 33/533; G01N 30/74

(52) U.S. Cl. CPC G01N 33/5743; G01N 2030/027; G01N 30/74; G01N 33/533

(57) ABSTRACT

The disclosure features compositions and methods that may be used to detect the presence of tumor cell-derived exosomes in a patient (e.g., a human patient) having cancer. The compositions and methods described herein may also be used to evaluate the patient's prognosis, as well as monitor the likelihood of the patient to benefit from therapy, such as immunotherapy. The disclosure also features antibodies that specifically bind chondroitin sulphate proteoglycan 4 (CSPG4), as well as antigen-antibody complexes containing the same.

### CROSS-REFERENCE TO RELATED APPLICATIONS

This application claims benefit of U.S. Provisional Application No. 62/856,195, filed Jun. 3,2019, the contents of which are incorporated herein by reference in their entirety.

### BACKGROUND OF THE INVENTION

While the mechanisms of this resistance to immune therapies are not understood, there is a growing awareness that the escape mechanisms cancer cells use to avoid recognition and destruction by the host immune system represent a major obstacle to successful immunotherapy. Multiple escape mechanisms used by tumors have been identified and characterized in the course of recent years.

Emerging evidence indicates that tumor cell-derived exosomes, a subpopulation of small extracellular vesicles (EVs), are an additional key player in the escape of tumor cells from immune surveillance (Whiteside et al., Clin Exp Immunol 189(3):259-267 (2017)). Exosomes are produced by both normal and malignant cells, but tumor cells produce them in excess (Whiteside et al., Clin Exp Immunol 189(3):259-267 (2017)). Exosomes circulate freely in body fluids, contain bioactive cargoes including proteins, lipids, and nucleic acids, and deliver their cargos to distant or near recipient cells.

Like other types of cancer cells, melanoma cells produce and release into body fluids various types of EVs. Exosomes are small EVs ranging in size from 30-150 nm that differ from other EVs by a unique biogenesis (Ruivo et al., Cancer Res 77(23):6480-6488 (2017)). Exosomes are formed in the endocytic compartment of parent cells by a vesiculation process involving reverse invagination of the multivesicular body (MVB) membrane and resulting in the formation of numerous intraluminal vesicles. When MVBs filled with intraluminal vesicles fuse with the surface membrane of the parent cell, the vesicles (i.e., exosomes) are released into the tissue space and disseminate throughout all body fluids (Hessvik et al., Cell Mol Life Sci 75(2):193-208 (2018)). Melanoma cells produce more exosomes than normal melanocytes, and melanoma patients' plasma contains increased levels of exosomes carrying a wide variety of cellular components, including proteins, lipids, and nucleic acids (Peinado et al., Nat Med 18(6):883-891 (2012)).

The molecular content of exosomes mimics that of their parent cell (Whiteside et al., Future Oncol 13(28):2583-2592 (2017)), and circulating exosomes are emerging as faithful tumor cell surrogates potentially useful as a liquid biopsy (Whiteside et al., Future Oncol 13(28):2583-2592 (2017)). Additionally, tumor cell-derived exosomes (TEX) carry a variety of biologically active molecules, including enzymes, growth factors, oncogenes, and signaling immunoregulatory proteins (Whiteside et al., Clin Exp Immunol 189(3):259-267 (2017)). Upon interaction with recipient cells, exosomes deliver their cargo to recipient cells and alter their functions (Mulcahy et al., J Extracell Vesicles. 3:24641 (2014)). In the TME, exosomes serve as a communication system between the tumor and the immune system (Kurywchak et al., Genome Med 10(1):23 (2018)). It has been shown that exosomes in melanoma patients' plasma contain an excess of immunosuppressive proteins (e.g., FasL, TGF-β, TRAIL, PD-L1) and inhibit functions of primary human immune cells in vitro and in vivo (Kurywchak et al., Genome Med 10(1):23 (2018); Chen et al., Nature 560(7718):382-386 (2018); Wieckowski et al., J Immunol 183(6):3720-3730 (2009); Sharma et al., Sci Rep 10(10):92 (2019)). Further, exosomes isolated from supernatants of melanoma cell lines, and containing only melanoma cell-derived exosomes, are highly enriched in inhibitory proteins and mediate strong immunosuppression (Wieckowski et al., J Immunol 183(6):3720-3730 (2009)).

Accordingly, there is a need in the art for methods that not only detect but also purify exosomes.

### SUMMARY OF THE INVENTION

In a first aspect, the disclosure features a method of detecting the presence of one or more tumor-cell derived exosomes in a patient having cancer, the method comprising contacting a sample obtained from the patient with an antibody, an antigen-binding fragment thereof, or a ligand that specifically binds chondroitin sulphate proteoglycan 4 (CSPG4); isolating the antibody, antigen-binding fragment, or ligand from the sample; and analyzing material bound to the antibody, antigen-binding fragment, or ligand for the presence of CSPG4, wherein a finding that the material bound to the antibody, antigen-binding fragment, or ligand comprises CSPG4 identifies the patient as having one or more tumor-cell derived exosomes. In some embodiments, a finding that the material bound to the antibody, antigen-binding fragment, or ligand comprises CSPG4 identifies the cancer as likely to resist detection and/or cell death by the patient's immune system. In some embodiments, a finding that the material bound to the antibody, antigen-binding fragment, or ligand comprises CSPG4 identifies the patient as likely to benefit from treatment with immunotherapy.

In another aspect, the disclosure features a method of determining whether a cancer in a patient suffering therefrom is likely to resist detection and/or cell death by the patient's immune system, the method comprising contacting a sample obtained from the patient with an antibody, an antigen-binding fragment thereof, or a ligand that specifically binds CSPG4; isolating the antibody, antigen-binding fragment, or ligand from the sample; and analyzing material bound to the antibody, antigen-binding fragment, or ligand for the presence of CSPG4, wherein a finding that the material bound to the antibody, antigen-binding fragment, or ligand comprises CSPG4 identifies the cancer as likely to resist detection and/or cell death by the patient's immune system. In some embodiments, a finding that the material bound to the antibody, antigen-binding fragment, or ligand comprises CSPG4 identifies the patient as having one or more tumor-cell derived exosomes. In some embodiments, a finding that the material bound to the antibody, antigen-binding fragment, or ligand comprises CSPG4 identifies the patient as likely to benefit from treatment with immunotherapy.

In another aspect, the disclosure features a method of determining whether a patient having cancer is likely to benefit from treatment with immunotherapy, the method comprising contacting a sample obtained from the patient with an antibody, an antigen-binding fragment thereof, or a ligand that specifically binds CSPG4; isolating the antibody, antigen-binding fragment, or ligand from the sample; and analyzing material bound to the antibody, antigen-binding fragment, or ligand for the presence of CSPG4, wherein a finding that the material bound to the antibody, antigen-binding fragment, or ligand comprises CSPG4 identifies the patient as likely to benefit from treatment with immunotherapy. In some embodiments, a finding that the material bound to the antibody, antigen-binding fragment, or ligand comprises CSPG4 identifies the patient as having one or more tumor-cell derived exosomes. In some embodiments, a finding that the material bound to the antibody, antigen-binding fragment, or ligand comprises CSPG4 identifies the cancer as likely to resist detection and/or cell death by the patient's immune system.

In some embodiments of any of the above aspects or embodiments of the disclosure, prior to contacting the sample with the antibody, antigen-binding fragment, or ligand, the sample is subjected to ultrafiltration through a filter having a pore size of about 0.2 μm. In some embodiments, prior to contacting the sample with the antibody, antigen-binding fragment, or ligand, the sample is subjected to differential centrifugation. In some embodiments, prior to contacting the sample with the antibody, antigen-binding fragment, or ligand, the sample is subjected to size exclusion chromatography.

In some embodiments, prior to contacting the sample with the antibody, antigen-binding fragment, or ligand the sample is prepared by subjecting the sample to differential centrifugation, subsequently subjecting the sample to ultrafiltration through a filter having a pore size of about 0.2 μm, and subsequently subjecting the sample to size exclusion chromatography.

In another aspect, the disclosure features a method of purifying one or more tumor-cell derived exosomes in a sample obtained from a patient having cancer, the method comprising subjecting the sample to differential centrifugation, ultrafiltration, and/or size exclusion chromatography; contacting the sample with an antibody, an antigen-binding fragment thereof, or a ligand that specifically binds CSPG4; and isolating the antibody, antigen-binding fragment, or ligand from the sample, wherein the method optionally comprises analyzing material bound to the antibody, antigen-binding fragment, or ligand for the presence of CSPG4.

In some embodiments of any of the above aspects or embodiments of the disclosure, the antibody, antigen-binding fragment, or ligand comprises a detectable label, and wherein the antibody, antigen-binding fragment, or ligand is isolated from the sample by contacting the antibody, antigen-binding fragment, or ligand with a compound that specifically binds the detectable label, and subsequently separating the compound from the sample. In some embodiments, the compound is immobilized to a surface, such as a bead. The bead may contain, for example, a polysaccharide, such as agarose. In some embodiments, the detectable label comprises biotin. In some embodiments, the compound comprises avidin or streptavidin.

In some embodiments of any of the above aspects or embodiments of the disclosure, the cancer is melanoma, glioma, head and neck cancer, mesothelioma, breast cancer, or ovarian cancer, optionally wherein the breast cancer is triple negative breast cancer. In some embodiments, the cancer is melanoma. In some embodiments, the cancer is a malignancy of orthopedic interest, such as a sarcoma (e.g., a soft tissue sarcoma, such as chordoma, chondrosarcoma, liposarcoma, or osteosarcoma).

In some embodiments, the one or more tumor cell-derived exosomes are melanoma cell-derived exosomes. In some embodiments, the one or more tumor cell-derived exosomes have a size of from about 30 to about 150 nm. In some embodiments, the one or more tumor cell-derived exosomes comprise one or more enzymes, growth factors, oncogenes and signaling immunoregulatory proteins. In some embodiments, the one or more tumor cell-derived exosomes comprise one or more immunosuppressive proteins. In some embodiments, the one or more immunosuppressive proteins comprise Fas ligand (FasL), transforming growth factor-beta (TGF-beta), TNF superfamily member 10 (TRAIL), and/or programmed death-ligand 1 (PD-L1).

In some embodiments, the antibody, antigen-binding fragment, or ligand specifically binds a first epitope on CSPG4, and wherein the material bound to the antibody, antigen-binding fragment, or ligand is analyzed for the presence of CSPG4 by contacting the material with an antibody, or an antigen-binding fragment thereof, that specifically binds a second epitope on CSPG4, wherein the first and second epitopes on CSPG4 are different from one another. In some embodiments, the first and second epitopes on CSPG4 do not share any overlapping amino acid residues. In some embodiments, the antibody or antigen-binding fragment that specifically binds the second epitope on CSPG4 comprises a detectable label. The detectable label on the antibody or antigen-binding fragment that specifically binds the second epitope on CSPG4 may contain, for example, a fluorophore. In some embodiments, the presence of CSPG4 is signaled by a finding of fluorescence at an emission wavelength characteristic of the fluorophore.

In some embodiments, the sample obtained from the patient contains blood plasma.

In another aspect, the disclosure features a method of determining whether a patient having cancer and that has been administered one or more therapeutic agents is benefiting from treatment with the one or more therapeutic agents, optionally wherein the one or more therapeutic agents comprise an immunotherapy, the method comprising contacting a sample obtained from the patient with an antibody, an antigen-binding fragment thereof, or a ligand that specifically binds CSPG4; isolating the antibody, antigen-binding fragment, or ligand from the sample; and analyzing material bound to the antibody, antigen-binding fragment, or ligand for the presence of CSPG4, wherein a finding that the quantity of CSPG4 in the sample has decreased relative to a previous measurement of CSPG4 in the patient is taken as an indication that the patient is benefiting from the treatment.

In another aspect, the disclosure features a method of treating a cancer in a patient in need thereof, the method comprising administering to the patient an immunotherapy, wherein the patient has been selected for treatment with an immunotherapy by the method of any one of above aspects or embodiments of the disclosure.

In some embodiments, the immunotherapy comprises an antibody, or an antigen-binding fragment thereof, that specifically binds an immune checkpoint protein. In some embodiments, the immune checkpoint protein is CTLA-4. In some embodiments, the immunotherapy comprises ipilimumab. In some embodiments, the immune checkpoint protein is PD-1. In some embodiments, the immunotherapy comprises pembrolizumab and/or nivolumab.

In some embodiments, the patient is a human.

In another aspect the disclosure features an antigen-antibody complex comprising a first antibody, or an antigen-binding fragment thereof, that specifically binds a first epitope on CSPG4; a second antibody, or an antigen-binding fragment thereof, that specifically binds a second epitope on CSPG4; and a CPSG4 proteoglycan, wherein the first and second epitopes on CSPG4 are different from one another. In some embodiments, the first and second epitopes on CSPG4 do not share any overlapping amino acid residues. In some embodiments, one or both of the first and second antibodies or antigen-binding fragments comprise a detectable label. In some embodiments, the first antibody or antigen-binding fragment comprises a detectable label comprising biotin. In some embodiments, the second antibody or antigen-binding fragment comprises a detectable label comprising a fluorophore.

In another aspect, the disclosure features a kit comprising a first antibody, or an antigen-binding fragment thereof, that specifically binds a first epitope on CSPG4; and a second antibody, or an antigen-binding fragment thereof, that specifically binds a second epitope on CSPG4, wherein the first and second epitopes on CSPG4 are different from one another, optionally wherein the first and second epitopes on CSPG4 do not share any overlapping amino acid residues. In some embodiments, one or both of the first and second antibodies or antigen-binding fragments comprise a detectable label. In some embodiments, the first antibody or antigen-binding fragment comprises a detectable label comprising biotin. In some embodiments, the kit further comprises a compound that specifically binds the detectable label of the first antibody or antigen-binding fragment. In some embodiments, the compound is immobilized to a surface. In some embodiments, the surface is a bead. In some embodiments, the bead comprises a polysaccharide. In some embodiments, the polysaccharide is agarose. In some embodiments, the compound comprises avidin or streptavidin. In some embodiments, the second antibody or antigen-binding fragment comprises a detectable label comprising a fluorophore. In some embodiments, the kit further comprises a filter suitable for ultrafiltration, wherein the filter has a pore size of about 0.2 μm. In some embodiments, the kit further comprises one or more size exclusion chromatography columns. In some embodiments, the kit further comprises a package insert instructing a user of the kit to perform the method of any of the above aspects or embodiments of the disclosure.

In another aspect, the disclosure features a use of an antibody, an antigen-binding fragment thereof, or a ligand that specifically binds CSPG4 in the manufacture of a kit for performing the method of any of the above aspects or embodiments of the disclosure.

As used herein, the term “antibody” refers to an immunoglobulin molecule that specifically binds to, or is immunologically reactive with, a particular antigen. The term “antibody” includes polyclonal, monoclonal, genetically engineered, and otherwise modified forms of immunoglobulin molecules, including, without limitation, chimeric antibodies, humanized antibodies, primatized antibodies, heteroconjugate antibodies (e.g., bi- tri- and quad-specific antibodies, diabodies, triabodies, and tetrabodies), and antigen-binding fragments of antibodies, including, for example, Fab′, F(ab′)₂, Fab, Fv, rIgG, and scFv fragments. Unless otherwise indicated, the term “monoclonal antibody” (mAb) is meant to include both intact molecules, as well as antibody fragments (such as, for example, Fab and F(ab′)₂ fragments) that are capable of specifically binding to a target protein.

The term “antigen-binding fragment,” as used herein, refers to one or more fragments of an antibody that retain the ability to specifically bind to a target antigen. The antigen-binding function of an antibody can be performed by fragments of a full-length antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (see, e.g., Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “benefit” refers to any clinical improvement in a patient's condition. Exemplary benefits in the context of a patient undergoing treatment for a disease or condition described herein include alleviation of symptoms, diminishment of extent of disease, stabilized state of disease, delay or slowing of disease progression, amelioration or palliation of disease state, and remission, whether partial or total. For example, in the context of a patient undergoing treatment for a cancer described herein, clinical indications of a benefit include, without limitation, a finding that the patient's rate of cancer cell growth has slowed or halted, a finding that the patient's rate of cancer metastasis has slowed or halted, a finding that the size of one or more tumors in the patient has decreased, a finding that the concentration of cancerous cells in a sample obtained from the patient has decreased relative to a previous measurement of the concentration of cancerous cells in a sample obtained from the patient, and a finding that the concentration of immunosuppressive proteins in a sample obtained from the patient has decreased relative to a previous measurement of the concentration of immunosuppressive proteins in a sample obtained from the patient.

As used herein, the term “cancer” refers to any member of a class of diseases or disorders characterized by uncontrolled division of cells and the ability of cells to invade other tissues, either by direct growth into adjacent tissue through invasion or by implantation into distant sites by metastasis. Metastasis is defined as the stage in which cancer cells are transported through the bloodstream or lymphatic system. Cancers are classified by the type of cell that the tumor resembles and, therefore, the tissue presumed to be the origin of the tumor. For example, carcinomas are malignant tumors derived from epithelial cells. This group represents the most common cancers, including the common forms of breast, prostate, lung, and colon cancer. Lymphomas and leukemias include malignant tumors derived from blood and bone marrow cells. Sarcomas are malignant tumors derived from connective tissue or mesenchymal cells. Mesotheliomas are tumors derived from the mesothelial cells lining the peritoneum and the pleura. Gliomas are tumors derived from glia, the most common type of brain cell. Germinomas are tumors derived from germ cells, normally found in the testicle and ovary. Cancers also include glioma, head and neck cancer, mesothelioma, triple-negative breast cancer, ovarian cancer, and malignancies of orthopedic interest, such as sarcomas (e.g., soft tissue sarcomas including chordoma, chondrosarcoma, liposarcoma, and osteosarcoma).

As used herein, the term “detectable label” refers to an atom, molecule, or complex that can signal the presence of a compound of interest. The presence of the compound may be signaled, for example, by a physical property of the detectable label, such as the ability of the detectable label to absorb or emit light at a particular wavelength. Exemplary detectable labels of this type include, without limitation, chromophores and fluorophores, such as chromophores and fluorophores known in the art. Other detectable labels signal the presence of a compound of interest by virtue of radioactivity. Examples of detectable labels of this type include atoms, molecules, and complexes containing one or more radioactive isotopes. The emission of radioactive particles from such isotopes can signal the presence of the compound of interest. Further examples of detectable labels include those that are specifically bound by a material, such as an antibody or antigen-binding fragment thereof, whose presence can be monitored by way of a molecular biology detection assay known in the art.

As used herein, the term “exosome” refers to a small membrane extracellular vesicle, such as from about 30 nm to about 300 nm in diameter, that is secreted from producing cells into the extracellular environment. The surface of an exosome contains a lipid bilayer formed from the membrane of the producing cell. The lumen of an exosome is topologically the same as the cytosol from the producing cell. Exosomes may contain proteins, nucleic acids (e.g., RNA), lipids, and/or carbohydrates of the producing cell. These molecules may be modified or added to the exosome after its release from the cell, either through natural processes or by experimental manipulation. Exosomes that are formed from a cancerous producing cell are referred to herein as “tumor cell-derived exosomes.”

As used herein, the term “immunosuppressive protein” refers to a protein that engages in a signal transduction pathway resulting in attenuation of a patient's immune response against an antigen, such as a tumor-associated antigen. Examples of immunosuppressive proteins include, without limitation, Fas ligand (FasL), transforming growth factor-beta (TGF-beta), TNF superfamily member 10 (TRAIL), and programmed death-ligand 1 (PD-L1).

As used herein, the terms “immunotherapy,” “immunotherapy agent,” and the like refer to a compound, such as an antibody or antigen-binding fragment thereof, that specifically binds an immune checkpoint protein (e.g., immune checkpoint receptor or ligand) and exerts an antagonistic effect on the receptor or ligand, thereby reducing or inhibiting the signal transduction of the receptor or ligand that would otherwise lead to a downregulation of the immune response. Immunotherapy agents include compounds, such as antibodies and antigen-binding fragments, that specifically bind receptors expressed on the surfaces of hematopoietic cells, such as lymphocytes (e.g., T cells), and suppressing the signaling induced by the receptor or ligand that would otherwise lead to tolerance towards an endogenous (“self”) antigen, such as a tumor-associated antigen. Immunotherapy agents may reduce the signaling induced by the receptor or ligand by, for example, 1%, 2%, 3%, 4%, 5%, 10%, 20%, 30%, 40%, 50%, 60%, 70%, 80%, 90%, 95%, 96%, 97%, 98%, 99%, 99.9%, or 100% relative to the signaling induced by the receptor or ligand exhibited in the absence of the immunotherapy agent. Exemplary assays that can be used to measure the extent of receptor or ligand signaling include, for example, ELISA techniques to measure protein expression alterations that are associated with a particular signal transduction pathway, as well as polymerase chain reaction (PCR)-based techniques, such as quantitative PCR, reverse-transcription PCR, and real-time PCR experiments useful for determining changes in gene expression associated with a particular signal transduction pathway, among others. Exemplary methods that can be used to determine whether an agent is an “immunotherapy agent” include the assays described in Mahoney et al., Cancer Immunotherapy, 14:561-584 (2015), the disclosure of which is incorporated herein by reference as it pertains to methods of monitoring the immunotherapy potential of a compound of interest. Examples of immunotherapy agents include antibodies and antigen-binding fragments thereof that specifically bind one or more of OX40L, TL1A, CD40L, LIGHT, BTLA, LAG3, TIM3, Singlecs, ICOS, B7-H3, B7-H4, VISTA, TMIGD2, BTNL2, CD48, KIR, LIR, LIR antibody, ILT, NKG2D, NKG2A, MICA, MICB, CD244, CSF1R, IDO, TGFβ, CD39, CD73, CXCR4, CXCL12, SIRPA, CD47, VEGF, and neuropilin. Additional example of immunotherapy agents include Targretin, Interferon-alpha, clobestasol, Peg Interferon (e.g., PEGASYS®), prednisone, Romidepsin, Bexarotene, methotrexate, Trimcinolone cream, anti-chemokines, Vorinostat, gabapentin, antibodies to lymphoid cell surface receptors and/or lymphokines, antibodies to surface cancer proteins, and/or small molecular therapies like Vorinostat. Particular examples of immunotherapy agents that may be used in conjunction with the compositions and methods described herein include anti-PD-1 antibodies and antigen-binding fragments thereof, anti-PD-L1 antibodies and antigen-binding fragments thereof, and anti-CTLA-4 antibodies and antigen-binding fragments thereof.

As used herein in the context of a cancer, the phrase “likely to resist detection and/or cell death by a patient's immune system” refers to the propensity of a cancer cell to avoid binding to an immune effector cell (e.g., a CD8+ cytotoxic T cell or a CD4+ helper T cell) and/or to evade the mounting of an immune attack by the patient's endogenous immune faculties. For example, a cancer cell is considered “likely to resist detection and/or cell death by a patient's immune system” if the cancer cell secretes one or more immunosuppressive proteins that attenuate the binding of an immune effector cell (e.g., a CD8+ cytotoxic T cell or a CD4+ helper T cell) and/or reduces the ability of the patient's immune system to kill the cancer cell.

As used herein, the term “patient” refers to an organism that receives treatment for a particular disease or condition as described herein, such as cancer. Examples of patients include mammals, such as humans, receiving treatment for diseases or conditions, for example, cell proliferation disorders, such as cancer.

As used herein, the phrase “specifically binds” refers to a binding reaction, which is determinative of the presence of an antigen among a heterogeneous population of proteins and other biological molecules that is recognized, for example, by an antibody or antigen-binding fragment thereof, with particularity. An antibody or antigen-binding fragment thereof that specifically binds to an antigen may non-covalently associate with the antigen with a K_{D} of, for example, less than 1 μM. For example, an antibody or antigen-binding fragment thereof that specifically binds to an antigen may bind to the antigen with a K_{D} of up to 100 nM (e.g., between 1 pM and 100 nM). A variety of immunoassay formats may be used to select antibodies specifically immunoreactive with a particular antigen. For example, solid-phase ELISA immunoassays may be used to select antibodies specifically immunoreactive with a desired antigen. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the terms “treat,” “treatment,” and the like refer to therapeutic treatment, in which the object is to prevent, decelerate, or lessen an undesired physiological change or disorder, such as the progression of a cell proliferation disorder (e.g., a cancer described herein). Clinical results indicative of successful treatment include, but are not limited to, alleviation of symptoms, diminishment of extent of disease, stabilized state of disease, delay or slowing of disease progression, amelioration or palliation of disease state, and remission, whether partial or total. Patients that are in need of treatment include those already having the condition or disorder, as well as those prone to developing the condition or disorder and those in which the condition or disorder is to be prevented.

The compositions and methods provide several useful clinical benefits. For example, using the compositions and methods described herein, one may detect not only the presence of tumor cell-derived exosomes in a cancer patient, but also quantitate the concentration of such exosomes in a sample obtained from the patient. As is described in further detail in the Examples below, the level of tumor cell-derived exosomes in a cancer patient informs the patient's disease prognosis, as well as the likelihood of the patient to respond to various forms of treatment, including immunotherapy. By detecting and quantitating the level of tumor cell-derived exosomes in a cancer patient, one can assess the patient's prognosis at an early stage, as well as monitor the patient's response to treatment over the course of the disease. In these ways, and in others described herein, the compositions and methods of the disclosure achieve significant and advantageous effects.

Other features and advantages of the invention will be apparent from the following description of the preferred embodiments thereof, and from the claims.

### BRIEF DESCRIPTION OF THE DRAWINGS

FIG. 1 shows methods for isolation of exosomes from plasma of patients with melanoma by size exclusion chromatography and separation of total exosomes recovered in fractions #3 and 4 into melanoma cell-derived exosomes (MTEX) and nonMTEX by immune capture with biotinylated anti-CSPG4 monoclonal antibodies (mAb). TME tumor microenvironment.

FIG. 2 shows the specificity of anti-CSPG4 monoclonal antibodies (mAb) clones TP4 and TP6 for detection of this antigen on melanoma cell-derived exosomes (MTEX) and nonMTEX. a,b Nearly all MTEX immunocaptured with clone TP4 mAb and detected with clone TP6 mAb are CSPG4(+); nonMTEX are negative for CSPG4. c,d Nearly all MTEX immunocaptured with clone TP6 and detected with clone TP4 mAb are CSPG4(+); nonMTEX are negative for CSPG4. The CSPG4-specific capture mAbs were used at the protein concentration of 3 μg, while anti-CD63 mAb for capture of nonMTEX was 2 μg. Note that dilu-tions of capture and detection mAbs are critical for the success of capture as well as detection of CSPG4+exosomes. a MTEX: capture antibody (clone VF1-TP41.2)—3 μg, detection antibody (clone TP61.5)—2.5 μg; b nonMTEX: capture antibody (anti-CD63)—2 μg, detection antibody (TP61.5)—0.75 μg; c MTEX: capture antibody (cloneTP61.5)—3 μg, detection anti-body (VF1-TP41.2)—2.5 μg; d nonMTEX: capture antibody (anti-CD63)—2 μg, detection antibody (VF1-TP41.2)—0.75 μg. RFI relative fluorescence index.

FIG. 3 shows chondroitin sulfate proteoglycan 4 (CSPG4) expression levels on Mel526 cells and on exosomes produced by MEL526 cells. Numbers indicate relative fluorescence index (RFI) values, which are nearly the same for cells and exosomes. a MEL526 cells, b MEL526 exosomes.

### DETAILED DESCRIPTION OF THE INVENTION

Below, we describe:(a) the characteristics of melanoma cell-derived exosomes (MTEX) separated by immunoaffinity capture from exosomes produced by non-malignant cells (non-MTEX); (b) the characteristics of the tumor antigen chondroitin sulfate proteoglycan 4 (CSPG4; Campoli et al., Crit Rev Immunol 24:267-296 (2004); Campoli et al., Adv Cancer Res 109:73-121 (2010)), which we used as a target to separate MTEX from non MTEX; and (c) the evidence supporting the role MTEX play in immune suppression in melanoma.

Accordingly, we describe the functional properties of the exosomes released from melanoma cells. In particular, we have detailed characteristics of the tumor antigen chondroitin sulfate proteoglycan 4 (CSPG4), which is used as a marker to separate exosomes released by melanoma cells from exosomes released by nonmalignant cells. Our results described herein are useful in view of the role of melanoma cell-derived exosomes in the escape of malignant cells from the host's immune system.

### Exosomes Serve as a Communication System Between the Tumor and the Immune System

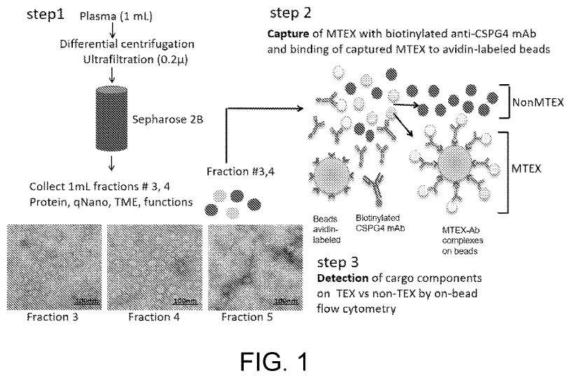

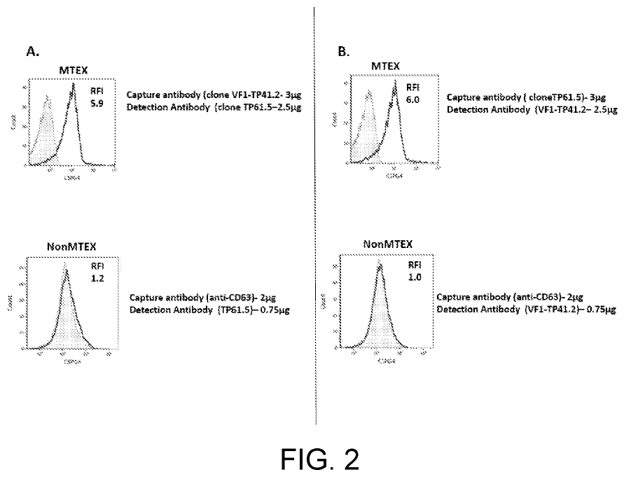

Melanoma patients' plasma contains exosomes produced by malignant and non-malignant cells. To analyze the phenotypic and functional characteristics of MTEX, we have developed an immunoaffinity-based method to separate them from nonMTEX. In this method, first the total exosome population is isolated from plasma using size exclusion chromatography (SEC), as described elsewhere (Hong et al., J Extracell Vesicles 5:29289 (2016)). Exosomes recovered in fractions #3,4 are separated using immune capture with the CSPG4-specific mAb into MTEX and nonMTEX, as also previously described (Sharma et al., J Extracell Vesicles 7(1):1435138 (2017)) and as shown in FIG. 1. The immune-captured MTEX are tested by on-bead flow cytometry for the expression of CSPG4 using a CSPG4-specific labeled mAb, which recognizes an epitope distinct and spatially distant from that recognized by the CSPG4-specific mAb used to capture MTEX. The CSPG4 antigen is expressed on MTEX, but is not detected on non-MTEX. Representative results generated by experiments performed with the CSPG4-specific mAb TP41.1 for capture and mAb TP61.1 for detection, which recognize distinct and spatially distant CSPG4 epitopes, are shown in FIG. 2. The data show that MTEX are positive (99%) and nonMTEX are negative for CSPG4. The protein cargo of successfully separated MTEX can now be further examined by on-bead flow cytometry using labeled mAbs that recognize melanoma/associated antigens (MAAs) or other proteins of interest in the MTEX cargo (Sharma et al., Sci Rep 10(10):92 (2019)). The MTEX and nonMTEX fractions can also be used for RNA or DNA extraction or can be co-incubated with various immune or non-immune cell types to determine their abilities to alter functions of recipient cells.

### Characteristics of Chondroitin Sulfate Proteoglycan 4

The rationale for selection of CSPG4 as a target antigen for immune capture of MTEX from melanoma patients' plasma is based on extensive evaluation of its expression on melanoma and normal human tissues. Like CD44, CSPG4 is a member of the CSPG family of cancer-associated proteins; CSPG4 is also known as a high molecular weight-melanoma-associated antigen (HMW-MAA), or neuron-glial antigen 2 (NG2; (Campoli et al., Crit Rev Immunol 24:267-296 (2004); Campoli et al., Adv Cancer Res 109:73-121 (2010))). The CSPG family members are key bioactive molecules that play a major role in tumor growth, migration, and neo-angiogenesis.

### Chondroitin Sulfate Proteoglycan 4 is Highly Expressed on Melanoma Cells

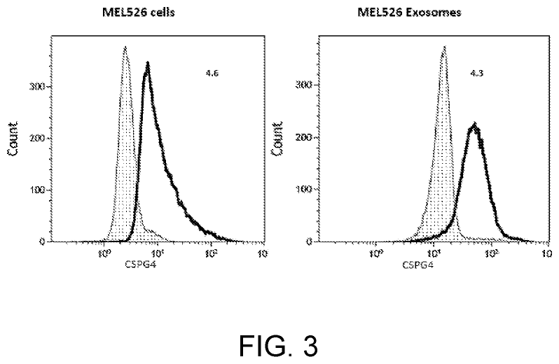

CSPG4 is highly expressed on melanoma cells in about 80% of primary and metastatic tumors with limited inter- and intralesional heterogeneity (Campoli et al., Crit Rev Immunol 24:267-296 (2004); Campoli et al., Adv Cancer Res 109:73-121 (2010)). It is expressed not only on differentiated melanoma cells, but also on malignant melanoma initiating cells (MMICs). The latter are defined as cells that can form spheres in vitro and are highly tumorigenic in immunodeficient mice. These cells express high levels of aldehyde dehydrogenase and are stained by an ABCB5-specific mAb RK1 (data not shown). In addition, as shown in FIG. 3, exosomes isolated from the spent medium of cultured melanoma cells by sequential differential centrifugation, filtration through a 2μ filter, and size exclusion chromatography are stained by CSPG4-specific mAbs with high intensity. As depicted in FIG. 3, exosomes were captured with biotinylated anti-63 mAb from supernatants of MEL526 cells, and on bead-cytometry was used for detection of CSPG4 on exosomes as described elsewhere (Sharma et al., J Extracell Vesicles 7(1):1435138 (2017)). Mel526 cells expressed high levels of surface CSPG4, and the exosomes these cells produce also carried high levels of CSPG4.

Data about the expression of CSPG4 in normal tissues are conflicting. In our own experience (Campoli et al., Crit Rev Immunol 24:267-296 (2004); Campoli et al., Adv Cancer Res 109:73-121 (2010); Wang et al., Cancer Res 71:7410-7422 (2011)), immuno-histochemical staining with mAbs that recognize distinct epitopes of CSPG4 has not detected expression of this antigen in any normal tissue with the exception of activated pericytes in the TME (Schlingemann et al., Am J Pathol 136:1393-1405 (1990); Maciag et al., Cancer Res 68:8066-8075 (2008)). Similar results have been reported by Morgan and his collaborators (Beard et al., J Immunother Cancer 2:25 (2014); Beard et al., Clin Cancer Res 19(18):4941-4950 (2013)) using different techniques. This conclusion has been corroborated by several lines of evidence. First, by analyzing 94 normal tissues from different organs with a reverse protein assay, we could detect CSPG4 expression above the threshold level in only two out of the four small-bowel samples tested. Second, no toxicity was detected: (a) in mice injected with large amounts of CSPG4-specific mAb cross-reacting with the mouse CSPG4 homologue (Wang et al., J Natl Cancer Inst 102:1496-1512 (2010)) and (b) in patients and dogs with melanoma as well as in rats with a chemically induced chondrosarcoma (Mittelman et al., Proc Natl Acad Sci USA 89:466-470 (1992); Riccardo et al., Clin Cancer Res 20:3753-3762 (2014); Léger et al., Int J Cancer 58(5):700-705 (1994)) who developed CSPG4-specific antibodies following immunizations with CSPG4 mimics. Lastly, CSPG4-specific CAR+T cells did not lyse various types of normal cells that are not stained by CSPG4-specific mAbs (Geldres et al., Clin Cancer Res 20:962-971 (2014)).

In contrast to our results summarized above, the data reported in the Protein Atlas, which have been obtained utilizing commercially available anti-CSPG4 antibodies, indicated that CSPG4 has a broad distribution in normal tissues. This conflicting evidence is likely caused by the lack of specificity of some of the commercial antibodies used to generate the data presented in the Protein Atlas. For instance, the rabbit antiserum provided by Sigma does not appear to be specific for CSPG4, since it recognizes a molecule with a molecular weight different from that of CSPG4 in Western blotting. Furthermore, the same antibody reacts with cells in which CSPG4 has been knocked out by CRISPR. The conflict that exists between our data and recommendations published in the Protein Atlas has led to confusion among investigators using commercial anti-CSPG4 mAbs for immune capture. We emphasize the specificity of our anti-CSPG4 mAbs for epitopes overexpressed on melanoma (or other tumor) cells and the lack of their reactivity with normal human tissues. Such specificity is useful for immune capture of CSPG4+ cells or exosomes. Some of the commercially available mAbs targeting CSPG4 fail to meet similar standards for tumor cell specificity and thus cannot be reliably used for immune capture of MTEX.

In sum, the tumor antigen CSPG4 is highly expressed on melanoma cells and is thus used as a marker to separate melanoma cell-derived exosomes (MTEX) from exosomes released form nonmalignant cells (nonMTEX).

A comparison of the phenotype and functional properties of MTEX and nonMTEX has shown that MTEX carry an abundance of immunosuppressive proteins and inhibit numerous functions of human primary T cells and natural killer cells. As a result, MTEX may promote tumor immune escape and tumor progression.

NonMTEX that are enriched in co-stimulatory proteins might stimulate immune cell activity.

Furthermore, we have demonstrated the separation of MTEX from nonMTEX fractions of exosomes in plasma of melanoma patients. The ability to perform this separation by immunoaffinity capture permits measuring the ratios of MTEX/nonMTEX in plasma of patients with metastatic melanoma. We have found that this ratio may vary from 20 to 60% (Sharma et al., Sci Rep 10(10):92 (2019)). In addition, we have been able to compare the phenotype and functional properties of MTEX and nonMTEX (Sharma et al., Sci Rep 10(10):92 (2019)). This comparison has shown that MTEX carry an abundance of immunosuppressive proteins and inhibit numerous functions of human primary T cells and natural killer (NK) cells ex vivo, as also described elsewhere (Wieckowski et al., J Immunol 183(6):3720-3730 (2009)). As a result, MTEX may promote tumor immune escape and tumor progression. By contrast, nonMTEX that are enriched in co-stimulatory proteins might stimulate immune cell activity (Sharma et al., Sci Rep 10(10):92 (2019); Sharma et al., J Extracell Vesicles 7(1):1435138 (2017)).

It is noteworthy that CSPG4 is also expressed on cancer cells in glioma, head and neck cancer, mesothelioma, triple-negative breast cancer, ovarian cancer, and malignancies of orthopedic interest (Campoli et al., Crit Rev Immunol 24:267-296 (2004); Campoli et al., Adv Cancer Res 109:73-121 (2010)). Therefore, the methodology we have developed for isolation of MTEX is applicable to other human malignancies.

From the foregoing description, one skilled in the art can easily ascertain the essential characteristics of this invention, can make various changes and modifications of the invention to adapt it to various usages and conditions. Thus, other embodiments are also within the claims.

The invention is also characterized by the following enumerated embodiments:

1. A method of detecting the presence of one or more tumor-cell derived exosomes in a patient having cancer, the method comprising:
a. contacting a sample obtained from the patient with an antibody, an antigen-binding fragment thereof, or a ligand that specifically binds chondroitin sulphate proteoglycan 4 (CSPG4);
b. isolating the antibody, antigen-binding fragment, or ligand from the sample; and
c. analyzing material bound to the antibody, antigen-binding fragment, or ligand for the presence of CSPG4;

wherein a finding that the material bound to the antibody, antigen-binding fragment, or ligand comprises CSPG4 identifies the patient as having one or more tumor-cell derived exosomes.

2. The method of embodiment 1, wherein a finding that the material bound to the antibody, antigen-binding fragment, or ligand comprises CSPG4 identifies the cancer as likely to resist detection and/or cell death by the patient's immune system.

3. The method of embodiment 1 or 2, wherein a finding that the material bound to the antibody, antigen-binding fragment, or ligand comprises CSPG4 identifies the patient as likely to benefit from treatment with immunotherapy.

4. A method of determining whether a cancer in a patient suffering therefrom is likely to resist detection and/or cell death by the patient's immune system, the method comprising:
a. contacting a sample obtained from the patient with an antibody, an antigen-binding fragment thereof, or a ligand that specifically binds CSPG4;
b. isolating the antibody, antigen-binding fragment, or ligand from the sample; and
c. analyzing material bound to the antibody, antigen-binding fragment, or ligand for the presence of CSPG4;

wherein a finding that the material bound to the antibody, antigen-binding fragment, or ligand comprises CSPG4 identifies the cancer as likely to resist detection and/or cell death by the patient's immune system.

5. The method of embodiment 4, wherein a finding that the material bound to the antibody, antigen-binding fragment, or ligand comprises CSPG4 identifies the patient as having one or more tumor-cell derived exosomes.

6. The method of embodiment 4 or 5, wherein a finding that the material bound to the antibody, antigen-binding fragment, or ligand comprises CSPG4 identifies the patient as likely to benefit from treatment with immunotherapy.

7. A method of determining whether a patient having cancer is likely to benefit from treatment with immunotherapy, the method comprising:
a. contacting a sample obtained from the patient with an antibody, an antigen-binding fragment thereof, or a ligand that specifically binds CSPG4;
b. isolating the antibody, antigen-binding fragment, or ligand from the sample; and
c. analyzing material bound to the antibody, antigen-binding fragment, or ligand for the presence of CSPG4;

wherein a finding that the material bound to the antibody, antigen-binding fragment, or ligand comprises CSPG4 identifies the patient as likely to benefit from treatment with immunotherapy.

8. The method of embodiment 7, wherein a finding that the material bound to the antibody, antigen-binding fragment, or ligand comprises CSPG4 identifies the patient as having one or more tumor-cell derived exosomes.

9. The method of embodiment 7 or 8, wherein a finding that the material bound to the antibody, antigen-binding fragment, or ligand comprises CSPG4 identifies the cancer as likely to resist detection and/or cell death by the patient's immune system.

10. The method of any one of embodiments 1-9, wherein, prior to contacting the sample with the antibody, antigen-binding fragment, or ligand, the sample is subjected to ultrafiltration through a filter having a pore size of about 0.2 μm.

11. The method of any one of embodiments 1-10, wherein, prior to contacting the sample with the antibody, antigen-binding fragment, or ligand, the sample is subjected to differential centrifugation.

12. The method of any one of embodiments 1-11, wherein, prior to contacting the sample with the antibody, antigen-binding fragment, or ligand, the sample is subjected to size exclusion chromatography.

13. The method of any one of embodiments 1-12, wherein, prior to contacting the sample with the antibody, antigen-binding fragment, or ligand the sample is prepared by:
a. subjecting the sample to differential centrifugation, subsequently
b. subjecting the sample to ultrafiltration through a filter having a pore size of about 0.2 μm, and subsequently
c. subjecting the sample to size exclusion chromatography.

14. A method of purifying one or more tumor-cell derived exosomes in a sample obtained from a patient having cancer, the method comprising:
a. subjecting the sample to differential centrifugation, ultrafiltration, and/or size exclusion chromatography;
b. contacting the sample with an antibody, an antigen-binding fragment thereof, or a ligand that specifically binds CSPG4; and
c. isolating the antibody, antigen-binding fragment, or ligand from the sample;

wherein the method optionally comprises analyzing material bound to the antibody, antigen-binding fragment, or ligand for the presence of CSPG4.

15. The method of any one of embodiments 1-14, wherein the antibody, antigen-binding fragment, or ligand comprises a detectable label, and wherein the antibody, antigen-binding fragment, or ligand is isolated from the sample by:
a. contacting the antibody, antigen-binding fragment, or ligand with a compound that specifically binds the detectable label, and subsequently
b. separating the compound from the sample.

16. The method of embodiment 15, wherein the compound is immobilized to a surface.

17. The method of embodiment 16, wherein the surface is a bead.

18. The method of embodiment 17, wherein the bead comprises a polysaccharide.

19. The method of embodiment 18, wherein the polysaccharide is agarose.

20. The method of any one of embodiments 15-19, wherein the detectable label comprises biotin.

21. The method of any one of embodiments 15-20, wherein the compound comprises avidin or streptavidin.

22. The method of any one of embodiments 1-21, wherein the cancer is melanoma, glioma, head and neck cancer, mesothelioma, breast cancer, or ovarian cancer, optionally wherein the breast cancer is triple negative breast cancer.

23. The method of embodiment 22, wherein the cancer is melanoma.

24. The method of any one of embodiments 1-3, 5, 6, and 8-23, wherein the one or more tumor cell-derived exosomes are melanoma cell-derived exosomes.

25. The method of any one of embodiments 1-3, 5, 6, and 8-24, wherein the one or more tumor cell-derived exosomes have a size of from about 30 to about 150 nm.

26. The method of any one of embodiments 1-3, 5, 6, and 8-25, wherein the one or more tumor cell-derived exosomes comprise one or more enzymes, growth factors, oncogenes and signaling immunoregulatory proteins.

27. The method of any one of embodiments 1-3, 5, 6, and 8-26, wherein the one or more tumor cell-derived exosomes comprise one or more immunosuppressive proteins.

28. The method of embodiment 27, wherein the one or more immunosuppressive proteins comprise Fas ligand (FasL), transforming growth factor-beta (TGF-beta), TNF superfamily member 10 (TRAIL), and/or programmed death-ligand 1 (PD-L1).

29. The method of any one of embodiments 1-28, wherein the antibody, antigen-binding fragment, or ligand specifically binds a first epitope on CSPG4, and wherein the material bound to the antibody, antigen-binding fragment, or ligand is analyzed for the presence of CSPG4 by contacting the material with an antibody, or an antigen-binding fragment thereof, that specifically binds a second epitope on CSPG4, wherein the first and second epitopes on CSPG4 are different from one another.

30. The method of embodiment 29, wherein the first and second epitopes on CSPG4 do not share any overlapping amino acid residues.

31. The method of embodiment 29 or 30, wherein the antibody or antigen-binding fragment that specifically binds the second epitope on CSPG4 comprises a detectable label.

32. The method of embodiment 31, wherein the detectable label on the antibody or antigen-binding fragment that specifically binds the second epitope on CSPG4 comprises a fluorophore.

33. The method of embodiment 32, wherein the presence of CSPG4 is signaled by a finding of fluorescence at an emission wavelength characteristic of the fluorophore.

34. The method of any one of embodiments 1-33, wherein the sample comprises blood plasma.

35. A method of determining whether a patient having cancer and that has been administered one or more therapeutic agents is benefiting from treatment with the one or more therapeutic agents, optionally wherein the one or more therapeutic agents comprise an immunotherapy, the method comprising:
a. contacting a sample obtained from the patient with an antibody, an antigen-binding fragment thereof, or a ligand that specifically binds CSPG4;
b. isolating the antibody, antigen-binding fragment, or ligand from the sample; and
c. analyzing material bound to the antibody, antigen-binding fragment, or ligand for the presence of CSPG4;

wherein a finding that the quantity of CSPG4 in the sample has decreased relative to a previous measurement of CSPG4 in the patient is taken as an indication that the patient is benefiting from the treatment.

36. A method of treating a cancer in a patient in need thereof, the method comprising administering to the patient an immunotherapy, wherein the patient has been selected for treatment with an immunotherapy by the method of any one of embodiments 3 and 6-34.

37. The method of any one of embodiments 3 and 6-36, wherein the immunotherapy comprises an antibody, or an antigen-binding fragment thereof, that specifically binds an immune checkpoint protein.

38. The method of embodiment 37, wherein the immune checkpoint protein is CTLA-4.

39. The method of embodiment 38, wherein the immunotherapy comprises ipilimumab.

40. The method of embodiment 37, wherein the immune checkpoint protein is PD-1.

41. The method of embodiment 40, wherein the immunotherapy comprises pembrolizumab and/or nivolumab.

42. The method of any one of embodiments 1-41, wherein the patient is a human.

43. An antigen-antibody complex comprising:
a. a first antibody, or an antigen-binding fragment thereof, that specifically binds a first epitope on CSPG4;
b. a second antibody, or an antigen-binding fragment thereof, that specifically binds a second epitope on CSPG4; and
c. a CPSG4 proteoglycan;

wherein the first and second epitopes on CSPG4 are different from one another.

44. The antigen-antibody complex of embodiment 43, wherein the first and second epitopes on CSPG4 do not share any overlapping amino acid residues.

45. The antigen-antibody complex of embodiment 43 or 44, wherein one or both of the first and second antibodies or antigen-binding fragments comprise a detectable label.

46. The antigen-antibody complex of any one of embodiments 43-45, wherein the first antibody or antigen-binding fragment comprises a detectable label comprising biotin.

47. The antigen-antibody complex of any one of embodiments 43-46, wherein the second antibody or antigen-binding fragment comprises a detectable label comprising a fluorophore.

48. A kit comprising:
a. a first antibody, or an antigen-binding fragment thereof, that specifically binds a first epitope on CSPG4; and
b. a second antibody, or an antigen-binding fragment thereof, that specifically binds a second epitope on CSPG4;

wherein the first and second epitopes on CSPG4 are different from one another, optionally wherein the first and second epitopes on CSPG4 do not share any overlapping amino acid residues.

49. The kit of embodiment 48, wherein one or both of the first and second antibodies or antigen-binding fragments comprise a detectable label.

50. The kit of embodiment 48 or 49, wherein the first antibody or antigen-binding fragment comprises a detectable label comprising biotin.

51. The kit of embodiment 49 or 50, wherein the kit further comprises a compound that specifically binds the detectable label of the first antibody or antigen-binding fragment.

52. The kit of embodiment 51, wherein the compound is immobilized to a surface.

53. The kit of embodiment 52, wherein the surface is a bead.

54. The kit of embodiment 53, wherein the bead comprises a polysaccharide.

55. The kit of embodiment 54, wherein the polysaccharide is agarose.

56. The kit of any one of embodiments 51-55, wherein the compound comprises avidin or streptavidin.

57. The kit of any one of embodiments 48-56, wherein the second antibody or antigen-binding fragment comprises a detectable label comprising a fluorophore.

58. The kit of any one of embodiments 48-57, wherein the kit further comprises a filter suitable for ultrafiltration, wherein the filter has a pore size of about 0.2 μm.

59. The kit of any one of embodiments 48-58, wherein the kit further comprises one or more size exclusion chromatography columns.

60. The kit of any one of embodiments 48-59, wherein the kit further comprises a package insert instructing a user of the kit to perform the method of any one of embodiments 1-42.

61. Use of an antibody, an antigen-binding fragment thereof, or a ligand that specifically binds CSPG4 in the manufacture of a kit for performing the method of any one of embodiments 1-42.

1. A method of detecting the presence of one or more tumor-cell derived exosomes in a patient having melanoma, the method comprising:
a. contacting a sample obtained from the patient with antibodies or antigen-binding fragments thereof which specifically bind distinct and spatially distant chondroitin sulphate proteoglycan 4 (CSPG4) epitopes on melanoma exosomes
wherein a finding that material bound to the melanoma exosomes, identifies the patient as having melanoma.

2. The method of claim 1, wherein a finding that the material bound to the melanoma exosomes identifies the melanoma as likely to resist detection and/or cell death by the patient's immune system.

3. The method of claim 1, wherein a finding that the material bound to the melanoma exosomes identifies the patient as likely to benefit from treatment with immunotherapy.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. The method of claim 1, wherein the patient is a human.

43. An antigen-antibody complex comprising:
a. a first antibody, or an antigen-binding fragment thereof, that specifically binds a first epitope on CSPG4;
b. a second antibody, or an antigen-binding fragment thereof, that specifically binds a second epitope on CSPG4; and
c. a CPSG4 proteoglycan;
wherein the first and second epitopes on CSPG4 are different from one another.

44. The antigen-antibody complex of claim 43, wherein the first and second epitopes on CSPG4 do not share any overlapping amino acid residues.

45. The antigen-antibody complex of claim 43, wherein one or both of the first and second antibodies or antigen-binding fragments comprise a detectable label.

46. The antigen-antibody complex of claim 43, wherein the first antibody or antigen-binding fragment comprises a detectable label comprising biotin.

47. The antigen-antibody complex of claim 43, wherein the second antibody or antigen-binding fragment comprises a detectable label comprising a fluorophore.

48. A kit comprising:
a. a first antibody, or an antigen-binding fragment thereof, that specifically binds a first epitope on CSPG4; and
b. a second antibody, or an antigen-binding fragment thereof, that specifically binds a second epitope on CSPG4;
wherein the first and second epitopes on CSPG4 are different from one another, optionally wherein the first and second epitopes on CSPG4 do not share any overlapping amino acid residues.

49. The kit of claim 48, wherein one or both of the first and second antibodies or antigen-binding fragments comprise a detectable label.

50. The kit of claim 48, wherein the first antibody or antigen-binding fragment comprises a detectable label comprising biotin.

51. The kit of claim 49, wherein the kit further comprises a compound that specifically binds the detectable label of the first antibody or antigen-binding fragment.

52. The kit of claim 51, wherein the compound is immobilized to a surface.

53. The kit of claim 52, wherein the surface is a bead.

54. The kit of claim 53, wherein the bead comprises a polysaccharide.

55. The kit of claim 54, wherein the polysaccharide is agarose.

56. The kit of claim 51, wherein the compound comprises avidin or streptavidin.

57. The kit of claim 48, wherein the second antibody or antigen-binding fragment comprises a detectable label comprising a fluorophore.

58. The kit of claim 48, wherein the kit further comprises a filter suitable for ultrafiltration, wherein the filter has a pore size of about 0.2 μm.

59. The kit of claim 48, wherein the kit further comprises one or more size exclusion chromatography columns.

60. (canceled)

61. (canceled)
